(19) United States
(12) Patent Application Publication
(10) Pub. No.: US 2006/0287397 A1
(43) Pub. Date: Dec. 21, 2006

(54) Dl-hydroxy-alkyl-phenylamides having anticonvulsive activity

(76) Inventor: Sergio Enrique Meza Toledo, Col. Nueva Santa Maria Mexico City (MX)

Correspondence Address: Thomas L Peterson, P O Box 5120, Springfield, VA, 22150, US

(21) Appl. No.: 10/512,490

(22) PCT Filed: Apr. 25, 2003
(86) PCT No.: PCT/MX03/00037

§ 371 Date: Aug. 28, 2006

(30) Foreign Application Priority Data
Apr. 26, 2002 (MX) PA/A/2002/004180

(51) Int. Cl. A61K 31/165; C07C 235/02

(52) U.S. Cl. 514/622; 564/170

(57) ABSTRACT

New anticonvulsant compounds were synthesized and they include the compounds: DL-2-hydroxy-2-(3′,5′-bistrifluoromethylphenyl)butyramide, DL-2-hydroxy-2-(4′-trifluoromethylphenyl)butyramide, DL-2-hydroxy-2-(3′,4′-dichlorophenyl)butyramide, DL-2-hydroxy-2-(3′-bromophenyl)butyramide, DL-2-hydroxy-2-(4′-bromophenyl)butyramide, DL-2-hydroxy-2-(3′-nitrophenyl)butyramide, DL-3-hydroxy-3-(3′,4′-dichlorophenyl)pentanamide, DL-3-hydroxy-3-(4′-bromophenyl)pentanamide, DL-4-hydroxy-4-(3′,4′-dichlorophenyl) hexanamide and DL-4-hydroxy-4-(4′-bromophenyl)hexanamide. They have a significant anticonvulsant activity against pentylenetetrazol-induced seizures as well as unexpected differences in anticonvulsant activity respect those of the non-halogenated compounds. The invention further provides methods for the synthesis of the DL-hydroxy-alkyl-phenyl amides as exemplified in the examples.

### TECHNICAL FIELD

The present invention relates to novel DL-hydroxy-alkyl-phenyl amides which have anticonvulsant activity, and to methods for preparation of such compounds.

### BACKGROUND OF THE INVENTION

The compounds DL-4-hydroxy-4-phenylhexanamide, DL-3-hydroxy-3-phenylpentanamide and DL-2-hydroxy-2-phenylbutyramide have a broad profile of anticonvulsant activity. They protect mice against seizures provoked by pentylenetetrazole, bicuculline, 4-aminopyridine, thiosemicarbazide and maximal electroshock. See Meza Toledo et al, Arzneim. Forsch. 40 (II), 1289 (1990). They also protect cats and rats against hippocampal kindling. See Solís et al, Arch. Neurocien. (Méx.) 1, 99 (1996). Furthermore, DL-3-hydroxy-3-phenylpentanamide protects rats against the γ-aminobutyric acid withdrawal syndrome, a model of focal epilepsy, which shows an extraordinary resistance to the classic antiepileptics. See Brailowsky et al, Epilepsy Res. 11, 167 (1992). The compound DL-3-hydroxy-3-phenylpentanamide also produces a significant decrease of focal spike activity in the genetic absence epilepsy rats of the Strasbourg model. See Brailowsky et al, cited above. Searching for more active anticonvulsants it was synthesized a new series of DL-hydroxy-alkyl-phenyl amides.

### DETAILED DESCRIPTION

DL-hydroxy-alkyl-phenyl amides which contain the trifluoromethyl, chlorine, bromine and nitro groups in the phenyl ring were synthesized. These compounds are known as DL-2-hydroxy-2-(3′,5′-bistrifluoromethylphenyl) butyramide (1), DL-2-hydroxy-2-(4′-trifluoromethylphenyl)butyramide (2), DL-2-hydroxy-2-(3′,4′-dichlorophenyl)butyramide (3), DL-2-hydroxy-2-(3′-bromophenyl)butyramide (4), DL-2-hydroxy-2-(4′-bromophenyl)butyramide (5), DL-2-hydroxy-2-(3′-nitrophenyl)butyramide (6), DL-3-hydroxy-3-(3′,4′-dichlorophenyl)pentanamide (7), DL-3-hydroxy-3-(4′-bromophenyl) pentanamide (8), DL-4-hydroxy-4-(3′,4′-dichlorophenyl)hexanamide (9) and DL-4-hydroxy-4-(4′-bromophenyl)hexanamide (10). They showed unexpected differences in anticonvulsant activity respect those of the non-halogenated compounds. The pentylenetetrazol test was used to evaluate the anticonvulsant activity of each compound. As it can be seen in Table 1, compounds 1, 3, 4, 5, 8 and 10 had an unusually high anticonvulsant activity, more than fivefold, sixfold or sevenfold that of the non-halogenated compounds. It is also noteworthy that the anticonvulsant activity of compounds 2, 6, 7 and 9 is twice, threefold or more than fourfold that of the non-halogenated compounds. This was particularly unexpected based on the nature of these compounds, and shows that substantial unexpected differences in activity exist for these compounds. Based on the protective effect of the compounds of the invention against pentylenetetrazol induced seizures, it may be expected that these compounds may be effective in the treatment of epilepsy of the absence type. In the following examples, melting points were determined with a Mettler apparatus. Infrared spectra were recorded on a Perkin Elmer Spectrum GX2000 spectrophotometer with ATR. ¹H-NMR spectra (270 MHz) and ¹³C-NMR spectra (100 MHz) were determined on a Jeol Eclipse apparatus and are reported as δ (ppm) values in CDCl₃ with TMS as the internal standard. Preparation of DL-2-hydroxy-2-(3′, 4′ or 5′ alkyl-phenyl)-butyramides Compounds 1, 2, 3, 4, 5 and 6 were prepared by condensation of the following ketones: 3′,5′-bis-trifluoromethylpropiophenone, 4′-trifluoromethylpropiophenone, 3′,4′-dichloropropiophenone, 3′-bromopropiophenone, 4′-bromopropiophenone and 3′-nitropropiophenone with trimethylsilyl cyanide, in the presence of zinc iodide, followed by partial hydrolysis under acidic conditions to obtain the amides 1, 2, 3, 4, 5 and 6.

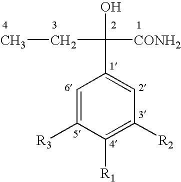

| Compound | R₁ | R₂ | R₃ |
|---|---|---|---|
| 1 | H | CF₃ | CF₃ |
| 2 | CF₃ | H | H |
| 3 | Cl | Cl | H |
| 4 | H | Br | H |
| 5 | Br | H | H |
| 6 | H | NO₂ | H |

### EXAMPLE 1

### Preparation of DL-2-hydroxy-2-(3′, 5′-bistrifluoromethylphenyl)butyramide (1)

To a solution of 2′,5′-bistrifluoromethylpropiophenone (10 g, 0.037 mol), methylene chloride (16 ml), under an atmosphere of nitrogen, was added zinc iodide (185 mg, 0.0005797 mol) and trimethylsilyl cyanide (4.29 g, 0.04316 mol). The reaction mixture was stirred at room temperature for 24 h and concentrated. The cooled residual oil was poured into 37% HCl (15 ml), then saturated with HCl_{(g)} and the mixture was allowed to stand at room temperature overnight. The precipitate was filtered off and crystallized from benzene to give 3.5 g (30%) of 1, m.p. 128-129° C.; IR: 1673, 3238, 3403 cm⁻¹; ¹H-NMR (CDCl₃/DMSO-d₆) δ: 0.98 (t, 3H, —CH₃); 2.05 (m, 2H, —CH₂—); 6.25 (bs, 1H, —NH₂); 7.0 (bs, 1H, —NH₂); 7.7 (s, 1H, H_{4′} phenyl), 8.1 (s, 2H, H_{2′,6′}, phenyl); ¹³C-NMR (CDCl₃) δ: 8.1 (C₄); 33.3 (C₃); 78.9 (C₂); 121.4 (C_{4′}); 123.8 (C_{CF3}); 126.1 (C_{2′,6′}); 131.3 (C_{3′,5′}); 146.6 (C_{1′}); 176.8 (C₁).

### EXAMPLE 2

### Preparation of DL-2-hydroxy-2-(4′-trifluoromethylphenyl)butyramide (2)

The compound 2 was prepared as described in the synthesis of 1, except that 4′-trifluoromethylpropiophenone (10 g, 0.0495 mol), zinc iodide (247 mg, 0.0007738 mol), methylene chloride (22 ml) and trimethylsilyl cyanide (5.73 g, 0.05774 mol) were used. Following the condensation, the cooled residual oil was poured into 37% HCl (15 ml) and the precipitate was filtered off and crystallized from benzene to obtain 10 g (81.8%) of 2, m.p. 129-130° C.; IR: 1678, 3178, 3463 cm⁻¹; ¹H-NMR (CDCl₃) δ: 0.69 (t, 3H, —CH₃); 1.78 (m, 1H, —CH₂—); 2.07 (m, 1H, —CH₂—); 5.02 (s, 1H, —OH), 6.35 (bs, 1H, —NH₂); 6.89 (bs, 1H, —NH₂); 7.34 (d, 2H, H_{2′,6′} phenyl), 7.58 (d, 2H, H_{3′,5′} phenyl); ¹³C-NMR (CDCl₃) δ: 8.2 (C₄); 32.5 (C₃); 79.0 (C₂); 122.7 (C_{CF3}); 125 (C_{3′,5′}); 126.5 (C_{2′,6′}); 129.3 (C_{4′}); 148 (C_{1′}); 177.2 (C₁).

### EXAMPLE 3

### Preparation of DL-2-hydroxy-2-(3′,4′-dichlorophenyl)butyramide (3)

The compound 3 was prepared as described in the synthesis of 1, except that 3′,4′-dichloropropiophenone (4.9 g, 0.024 mol), zinc iodide (121 mg, 0.000379 mol), methylene chloride (15 ml) and trimethylsilyl cyanide (2.88 g, 0.029 mol) were used. Following the condensation, the cooled residual oil was poured into 37% HCl (10 ml) and the precipitate was filtered off and crystallized from benzene to obtain 1.4 g (23.5%) of 3, m.p. 102-103° C.; IR: 1662, 3190, 3480 cm⁻¹; ¹H-NMR (CDCl₃) δ: 0.85 (t, 3H, —CH₃); 1.94 (m, 1H, —CH₂—); 2.22 (m, 1H, —CH₂—); 3.4 (s, 1H, —OH); 5.9 (bs, 1H, —NH₂); 6.79 (bs, 1H, —NH₂); 7.45 (s, 2H, H_{2′,6′} phenyl), 7.77 (s, 1H, H_{5′} phenyl); ¹³C-NMR (CDCl₃) δ: 8.03 (C₄), 32.72 (C₃), 78.99 (C₂), 125.52 (C_{6′}), 128.15 (C_{5′}), 130.64 (C_{2′}), 132.30 (C_{3′}), 132.88 (C_{4′}), 142.78 (C_{1′}), 176.45 (C₁).

### EXAMPLE 4

### Preparation of DL-2-hydroxy-2-(3′-bromophenyl)butyramide (4)

The compound 4 was prepared as described in the synthesis of 1, except that 3′-bromopropiophenone (17.12 g, 0.0807 mol), zinc iodide (400 mg, 0.001253 mol), methylene chloride (34 ml) and trimethylsilyl cyanide (12.46 g, 0.1255 mol) were used. Following the condensation, the cooled residual oil was poured into 37% HCl (15 ml) and the precipitate was filtered off and crystallized from benzene to obtain 5.5 g (53.3%) of 4, m.p. 78-79° C.; IR: 1668, 3259, 3454 cm⁻¹; ¹H-NMR (CDCl₃) δ: 0.90 (t, 3H, —CH₃); 1.9 (m, 1H, —CH₂—); 2.2 (m, 1H, —CH₂—); 3.8 (s, 1H, —OH); 6.1 (bs, 1H, —NH₂); 6.8 (bs, 1H, —NH₂); 7.2 (m, 1H, H_{5′}, phenyl), 7.4 (m, 1H, H_{6′}, phenyl), 7.6 (s, 1H, H_{2′}, phenyl), 7.8 (m, 1H, H_{4′}, phenyl); ¹³C-NMR (CDCl₃) δ: 8.1 (C₄); 32.5 (C₃); 79 (C₂); 123 (C_{3′}); 124.7 (C_{6′}); 129.1 (C_{4′}); 130.3 (C_{5′}); 131.2 (C_{2′}); 145 (C_{1′}); 177 (C₁).

### EXAMPLE 5

### Preparation of DL-2-hydroxy-2-(4′-bromophenyl)butyramide (5)

The compound 5 was prepared as described in the synthesis of 1, except that 4′-bromopropiophenone (9 g, 0.0422 mol), zinc iodide (200 mg, 0.000626 mol), methylene chloride (17 ml) and trimethylsilyl cyanide (6.23 g, 0.0627 mol) were used. Following the condensation, the cooled residual oil was poured into 37% HCl (15 ml) and the precipitate was filtered off and crystallized from benzene to obtain 7.7 g (70.8%) of 5, m.p. 148-149° C.; IR (KBr): 1673, 3229, 3454 cm⁻¹; ¹H-NMR (CDCl₃) δ: 0.50 (t, 3H, —CH₃); 1.55 (m, 1H, —CH₂—); 1.83 (m, 1H, —CH₂—); 5.12 (s, 1H, —OH); 6.27 (bs, 1H, —NH₂); 6.67(bs, 1H, —NH₂); 7.04 (d, 2H, H_{2′,6′}, phenyl), 7.13 (d, 2H, H_{3′,5′}, phenyl); ¹³C-NMR (CDCl₃) δ 7.88 (C₄); 31.9 (C₃); 78.2 (C₂); 120.69 (C_{4′}); 127.56 (C_{2′,6′}); 130.54 (C_{3′,5′}); 142.83 (C_{1′}); 176.73 (C₁).

### EXAMPLE 6

### Preparation of DL-2-hydroxy-2-(3′-nitrophenyl)butyramide (6)

The compound 6 was prepared as described in the synthesis of 1, except that 3′-nitropropiophenone (7 g, 0.039 mol), zinc iodide (195 mg, 0.00061 mol), methylene chloride (30 ml) and trimethylsilyl cyanide (4.64 g, 0.0468 mol) were used. Following the condensation, the cooled residual oil was poured into 37% HCl (15 ml) and the precipitate was filtered off and crystallized from benzene to obtain 3.4 g (38.9%) of 6, m.p. 99-101° C.; IR: 1520, 1650, 3270, 3410 cm⁻¹; ¹H-NMR (CDCl₃) δ: 0.43 (t, 3H, —CH₃), 1.53 (q, 1H, —CH₂Me), 1.81 (q, 1H, —CH₂Me), 5.45 (s, 1H, —OH), 6.41 (bs, 1H, NH₂), 6.72 (bs, 1H, —NH₂), 7.05 (t, 1H, H_{5′} phenyl), 7.60 (q, 2H, H_{4′,6′} phenyl), 8.05 (d, 2H, H_{2′} phenyl); ¹³C-NMR (CDCl₃) δ 7.86 (C₄), 32.4 (C₃), 78.34 (C₂), 120.79 (C_{2′}), 122.00 (C_{4′}), 128.8 (C_{5′}), 132.3 (C_{6′}), 145.7 (C_{1′}), 147.9 (C_{3′}), 176.5 (C₁).

### Preparation of DL-3-hydroxy-3-(3′,4′-dichlorophenyl or 4′-bromophenyl)-pentanamides

Compounds 7 and 8 were prepared by condensation of the following ketones: 3′,4′-dichloropropiophenone and 4′-bromopropiophenone with ethyl bromoacetate in the presence of zinc, via the hydroxyesters, which were reacted either trimethylaluminium plus liquid ammonia or ammonia to give 7 and 8.

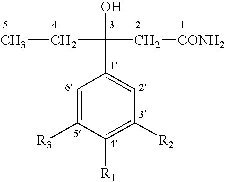

| Compound | R₁ | R₂ | R₃ |
|---|---|---|---|
| 7 | Cl | Cl | H |
| 8 | Br | H | H |

### EXAMPLE 7

### Preparation of DL-3-hydroxy-3-(3′,4′-dichlorophenyl)pentanamide (7)

### A. Preparation of DL-ethyl-3-hydroxy-3-(3′,4′-dichlorophenyl)pentanoate

To a stirred solution of 3′,4′-dichloropropiophenone (5 g, 0.0246 mol), ethyl bromoacetate (5.8 g, 0.0348 mol), benzene (10 ml) and diethyl ether (3 ml) was added activated zinc (2 g, 0.0306 mol). The reaction mixture was heated at reflux for 5 h and then hydrolyzed with 10% H₂SO₄ (20 ml). The organic layer was separated and washed sequentially with 5% H₂SO₄ (2×10 ml), 10% Na₂CO₃ (10 ml), 5% H₂SO₄ (10 ml) and water (2×10 ml). The benzene layer was separated and the combined acid solutions were extracted with diethyl ether (2×10 ml). The ether extracts and the organic layer separated initially were combined, dried over MgSO₄, filtered and concentrated. The residue was fractionated at reduced pressure to give 4.6 g (64.2%) of DL-ethyl-3-hydroxy-3-(3′,4′-dichlorophenyl)pentanoate, b.p. 138-140° C./30 mm Hg; IR: 1680, 3470 cm⁻¹; ¹H-NMR (CDCl₃) δ: 0.75 (t, 3H, —CH₂—CH₃), 1.13 (t, 3H, —COOCH₂—CH₃), 1.75 (m, 2H, —CH₂Me), 2.78 (d,d, 2H, —CH₂—), 4.03 (d,d, 2H, —COOCH₂Me), 4.41 (s, 1H, —OH), 7.20 (d,d, 1H, H_{6′} phenyl), 7.37 (d, 1H, H_{2′} phenyl), 7.50 (s, 1H, H_{5′} phenyl); ¹³C-NMR (CDCl₃) δ 8.01 (C₅), 13.98 (C_{CH3}), 35.71 (C₄), 44.50 (C₂), 60.90 (C_{—OCH2-}), 74.72 (C₃), 124.75 (C_{5′}), 127.01 (C_{2′}), 127.62 (C_{6′}), 130.01 (C_{3′}), 130.70 (C_{4′}), 145.84 (C_{1′}), 172.63 (C₁).

### B. Preparation of DL-3-hydroxy-3-(3′,4′-dichlorophenyl)pentanamide (7)

To a cooled mixture of methylene chloride (74 ml) and liquid ammonia (1 ml), under an atmosphere of nitrogen, was added a solution of trimethylaluminum (1.08 g, 0.015 mol) in hexanes. The mixture was stirred for 30 min and DL-ethyl-3-hydroxy-3-(3′,4′-dichlorophenyl)pentanoate (4.3 g, 0.01478 mol) was added dropwise. The solution was heated at reflux for 48 h, then it was cooled and 0.5 N HCl (30 ml) was added. The organic layer was separated and the aqueous solution was extracted with three 30 ml portions of diethyl ether. The combined organic layers were washed with brine (100 ml), dried over NaSO₄, filtered and concentrated to obtain 1.2 g (31.0%) of 7, m.p. 99-100° C.; IR: 1660, 3170, 3370 cm⁻¹; ¹H-NMR (CDCl₃) δ: 0.75 (t, 3H, —CH₃), 1.81 (m, 2H, —CH₂Me), 2.67 (t, 2H, —CH₂—), 5.23 (s, 1H, —OH), 5.74 (bs, 2H, —NH₂), 7.22 (t, 1H, H_{6′} phenyl), 7.37 (d, 1H, H_{2′} phenyl), 7.50 (s, 1H, H_{5′} phenyl); ¹³C-NMR (CDCl₃) δ: 7.58 (C₅), 35.67 (C₄), 45.34 (C₂), 76.72 (C₃), 124.78 (C_{6′}), 127.59 (C_{2′}), 130.13 (C_{5′}), 130.75 (C_{3′}), 132.38 (C_{4′}), 145.89 (C_{1′}), 174.30 (C₁).

### EXAMPLE 8

### Preparation of DL-3-hydroxy-3-(4′-bromophenyl)pentanamide (8)

### A. Preparation of DL-ethyl-3-hydroxy-3-(4′-bromophenyl)pentanoate

The compound DL-ethyl-3-hydroxy-3-(4′-bromophenyl)pentanoate was prepared as described in the synthesis of DL-ethyl-3-hydroxy-3-(3′,4′-dichlorophenyl)pentanoate, except that 4′-bromopropiophenone (25 g, 0.117 mol), ethyl bromoacetate (13.41 g, 0.08 mol), benzene (40 ml), diethyl ether (10 ml) and activated zinc (8.18 g, 0.125 mol) were added. Following the hydrolysis and extraction, the residual oil was fractionated at reduced pressure to give 17 g (70.6%) of DL-ethyl-3-hydroxy-3-(4′-bromophenyl)pentanoate, b.p. 193-195° C./12 mm Hg; IR: 1715, 3480 cm⁻¹.

### B. Preparation of DL-3-hydroxy-3-(4′-bromophenyl)pentanamide (8)

DL-ethyl-3-hydroxy-3-(4′-bromophenyl)pentanoate (10 g, 0.033 mol), ethanol (50 ml) and 28% aqueous ammonia (100 ml) were cooled at 0° C. and saturated with ammonia gas. The flask was closed with a rubber stopper and held at room temperature for 30 days. Then the mixture was cooled, the stopper removed and sodium chloride (10 g) was added. The reaction mixture was extracted with two 50 ml portions of diethyl ether and the combined ether extracts were dried over Na₂SO₄ and were evaporated to dryness. The residue was crystallized from water to give 4 g (44.2%) of 8, m.p. 118-119° C.; IR: 1660, 3160, 3380 cm⁻¹; ¹H-NMR (CDCl₃) δ: 0.73 (t, 3H, —CH₃), 1.77 (q, 2H, —CH₂Me), 2.66 (s, 2H, —CH₂—), 5.07 (s, 1H, —OH), 5.71 (bs, 2H, —CONH₂), 7.26 (d, 2H, H_{2′,6′} phenyl), 7.41 (d, 2H, H_{3′,5′} phenyl); ¹³C-NMR (CDCl₃) δ: 7.56 (C₅), 35.7 (C₄), 44.8 (C₂), 74.93 (C₃), 119.92 (C_{4′}), 127.34 (C_{2′,6′}), 130.61 (C_{3′,5′}), 145.08 (C_{1′}), 174.65 (C₁).

### Preparation of DL-4-hydroxy-4-(3′,4′-dichlorophenyl or 4′-bromophenyl)-hexanamides

Compounds 9 and 10 were obtained by condensation of the following ketones: 3′,4′-dichloropropiophenone and 4′-bromopropiophenone with diethyl succinate, in the presence of sodium hydride, followed by cyclization under acidic conditions to yield the lactones. Treatment of the latter either trimethylaluminium plus liquid ammonia or ammonia gave 9 and 10.

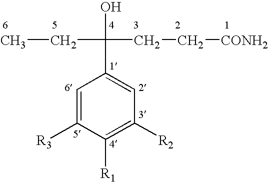

| Compound | R₁ | R₂ | R₃ |
|---|---|---|---|
| 9 | Cl | Cl | H |
| 10 | Br | H | H |

### EXAMPLE 9

### Preparation of DL-4-hydroxy-4-(3′,4′-dichlorophenyl)hexanamide (9)

### A. Preparation of DL-5-ethyl-5-(3′,4′-dichlorophenyl)butyrolactone

A mixture of 3′,4′-dichloropropiophenone (55 g, 0.27 mol), benzene (250 ml), diethyl succinate (141.4 g, 0.812 mol), sodium hydride (19.5 g, 0.81 mol) and absolute ethanol (4 ml), under an atmosphere of nitrogen, was stirred for 135 min. The reaction was quenched by the sequential dropwise addition of glacial acetic acid (44 ml) and water (40 ml). The organic layer was separated and the aqueous solution was extracted with four 50 ml portions of diethyl ether. The organic layers were combined and washed with 5% Na₂CO₃ (2×50 ml) solution. This solution, after acidification with 98% H₂SO₄, was re-extracted with two 50 ml portions of diethyl ether. The two organic layers were combined, dried over MgSO₄, filtered and concentrated to provide an oily residue (36 g). This oily crude product, glacial acetic acid (167 ml), 48% HBr (111 ml) and water (56 ml) were heated at reflux for 26 h. The reaction mixture was concentrated under vacuum and extracted with four 50 ml portions of diethyl ether. The organic layers combined were washed with a 5% NaHCO₃ solution. The alkaline solution was acidified with 98% H₂SO₄ and was extracted with diethyl ether. The combined organic layers were concentrated and the acidic materials separated. This oily crude product, glacial acetic acid (45 ml), 48% HBr (30 ml) and water (15 ml) were heated at reflux for 40 h and the reaction product was treated as previously. The neutral fractions of the second treatment were combined with the ethereal solution of the original reaction and washed with brine, dried over MgSO₄, filtered and concentrated. The residue was fractionated under reduced pressure to give 15 g (21.45%) of DL-5-ethyl-5-(3′,4′-dichlorophenyl)butyrolactone, b.p. 172-173° C./15 mm Hg. IR: 1773 cm⁻¹.

### B. Preparation of DL-4-hydroxy-4-(3′,4′-dichlorophenyl)hexanamide (9)

To a cooled mixture of methylene chloride (300 ml) and liquid ammonia (4.8 ml), under an atmosphere of nitrogen, was added a solution of trimethylaluminum (8.3 g, 0.1152 mol) in hexanes. The mixture was stirred for 30 min and then a solution of DL-5-ethyl-5-(3′,4′-dichlorophenyl)butyrolactone (7.5 g, 0.0289 mol) in methylene chloride (150 ml) was added dropwise. The solution was heated at reflux for 48 h, then it was cooled and 0.5 N HCl (227 ml) was added. The organic layer was separated and the aqueous solution was extracted with three 100 ml portions of diethyl ether. The combined organic layers were washed with brine (300 ml), dried over NaSO₄, filtered and concentrated. The residue was crystallized from hexane-methylene chloride to give 2.0 g (25.07%) of 9, m.p. 106-107° C.; IR: 1674, 3179, 3342 cm⁻¹; ¹H-NMR (CDCl₃) δ: 0.71 (t, 3H, —CH₃), 1.73-1.82 (m, 2H, —CH₂Me), 1.95-2.2 (m, 4H, —CH₂—CH₂—), 5.11 (s, 1H, —OH), 6.41 (bs, 1H, —NH₂), 6.98 (bs, 1H, —NH₂), 7.14-7.39 (m, 3H, H_{2′,5′,6′} phenyl); ¹³C-NMR (CDCl₃) δ: 12.88 (C₆), 35.16 (C₅), 41.16 (C₃), 42.38 (C₂), 80.52 (C₄), 130.60 (C_{6′}), 130.93 (C_{4′}), 132.33 (C_{2′}), 132.80 (C_{5′}), 136.38 (C_{3′}), 149.96 (C_{1′}), 181.99 (C₁).

### EXAMPLE 10

### Preparation of DL-4-hydroxy-4-(4′-bromophenyl)hexanamide (10)

### A. Preparation of DL-5-ethyl-5-(4′-bromophenyl)butyrolactone

The compound DL-5-ethyl-5-(4′-bromophenyl)butyrolactone was prepared as described in the synthesis of DL-5-ethyl-5-(3′,4′-dichlorophenyl)butyrolactone, except that 4′-bromopropiophenone (43.5 g, 0.324 mol), benzene (225 ml), diethyl succinate (130.65 g, 0.75 mol), sodium hydride (18 g, 0.75 mol) and absolute ethanol (3.65 ml) were added. Following the acidification and extraction, the oily residue was heated under reflux with CH₃COOH/HBr. The combination of the neutral fractions of the second treatment with CH₃COOH/HBr with the ethereal solution of the original reaction gave an oily residue which was fractionated under reduced pressure to give 18 g (41.4%) of DL-5-ethyl-5-(4′-bromophenyl)butyrolactone, b.p. 160-161° C./20 mm Hg; IR: 1773 cm⁻¹.

### B. Preparation of DL-4-hydroxy-4-(4′-bromophenyl)hexanamide (10)

DL-5-ethyl-5-(4′-bromophenyl)butyrolactone (18 g, 0.0669 mol), ethanol (80 ml) and 28% aqueous ammonia (80 ml) were cooled at 0° C. and saturated with ammonia gas. The flask was closed with a rubber stopper and held at room temperature for 21 days. Then the mixture was cooled, the stopper removed and sodium chloride (7.5 g) was added. The reaction mixture was extracted with three 100 ml portions of diethyl ether and the combined ether extracts were dried over Na₂SO₄ and were evaporated to dryness. The residue was crystallized from water to give 5.3 g (27.7%) of 10, m.p. 122-123° C.; IR: 1682, 3197, 3384 cm⁻¹; ¹H-NMR (CDCl₃) δ: 0.71-0-77 (t, 3H, —CH₃), 1.75-1.84 (q, 2H, —CH₂Me), 2.01-2.23 (m, 4H, —CH₂—CH₂—), 3.90 (s, 1H, —OH), 5.41 (bs, 2H, —NH₂), 7.23 (d, 2H, H_{2′,6′} phenyl), 7.42 (d, 2H, H_{3′,5′} phenyl); ¹³C-NMR (CDCl₃) δ: 7.80 (C₆); 30.36 (C₅); 36.56 (C₃), 36.93 (C₂), 76.60 (C₄); 120.37 (C_{4′}); 127.64 (C_{2′,6′}); 131.25 (C_{3′,5′}); 144.76 (C_{1′}); 176.72 (C₁).

### EXAMPLE 11

### Anticonvulsant Activity

A. General Methods

Male albino mice CF-1 strain (National Institute of Virology, Mexico City) weighing 18-25 g were housed in groups of 20, at room temperature (20-24° C.), with tap water and food (pellet type Blue Bonnet) ad libitum, with a 12-h light-dark cycle. DL-2-hydroxy-2-phenyl butyramide, DL-3-hydroxy-3-phenyl pentanamide, DL-4-hydroxy-4-phenyl hexanamide and pentylenetetrazol were dissolved in water and compounds 1-10 were dissolved in a 30% polyethyleneglycol-400 solution. All the compounds used were administered intraperitoneally (i.p.). The convulsant dose of pentylenetetrazol inducing seizures and death in 100% of mice (CD₁₀₀) was determined and used in the test. CD₁₀₀ value obtained was 100 mg kg¹. To determine the dose-response curves five separate groups of 10-20 mice were dosed i.p. with the test compounds, and pentylenetetrazol, 100 mg kg⁻¹, i.p., was administered at 30 min after. The suppression of clonic seizures and death was considered the end point of the test. The vehicle was inactive in all the test procedures. The ED₅₀ and 95% confidence intervals were calculated by the method of Litchfield and Wilcoxon (Table 1). See Litchfield et al., J. Pharmacol. Exp. Ther. 96, 99 (1949).

TABLE 1

| | ED₅₀ (mgkg⁻¹) | |
|---|---|---|
| COMPOUND | PTZ ^{a)} | |
| 1 | 10 | (7-13)^{b)} |
| 2 | 23 | (20-27) |
| 3 | 10 | (8-13) |
| 4 | 8 | (6-12) |
| 5 | 8 | (6-11) |
| 6 | 36 | (31-42) |
| 7 | 16 | (13-20) |
| 8 | 8 | (6-12) |
| 9 | 12 | (9-17) |
| 10 | 9 | (6-13) |
| DL-2-hydroxy-2- | 58 | (50-68) |
| Phenyl butyramide | | |
| DL-3-hydroxy-3- | 49 | (42-57) |
| Phenyl pentanamide | | |
| DL-4-hydroxy-4- | 55 | (49-62) |
| Phenylhexanamide | | |

^{a)} Seizures induced by pentylenetetrazol (PTZ) in mice.
^{b)}95% confidence interval.

1-20. (canceled)

21. A method for the synthesis of DL-2-hydroxy-2-(3′, 4′ or 5′ alkyl-phenyl) butyramides which comprises:
A) Reacting an alkylated propiophenone in the phenyl ring with trimethylsilyl cyanide, in the presence of zinc iodide, followed by partial hydrolysis under acidic conditions to obtain the DL-2-hydroxy-2-(3′, 4′ or 5′ alkylphenyl)butyramides.

22. A method for the synthesis of DL-3-hydroxy-3-(3′,4′-dichlorophenyl or 4′-bromophenyl)pentanamides which comprises:
A) Reacting either 3′,4′-dichloropropiophenone or 4′-bromopropiophenone with ethyl bromoacetate in the presenc of zinc to obtain DL-5-ethyl-3-(3′,4′-dichlorophenyl or 4′-bromophenyl)pentanoates; and
B) Transforming the ester group of said pentanoates into an —NH₂ group by reacting the ester group either trimethylaluminium plus liquid ammonia or ammonia to form DL-3-hydroxy-3-(3′,4′-dichlorophenyl or 4′-bromophenyl)-pentanamides.

23. A method for the synthesis of DL-4-hydroxy-4-(3′,4′-dichlorophenyl or 4′-bromophenyl)hexanamides which comprises:
A) Reacting either 3′,4′-dichloropropiophenone or 4′-bromopropiophenone with diethyl succinate in the presence of sodium hydride, followed by cyclization under acidic conditions to yield DL-5-ethyl-5-(3′,4′-dichlorophenyl or 4′-bromophenyl) butyrolactones; and
B) Transforming the lactones either trimethylaluminium plus liquid ammonia or ammonia to give DL-4-hydroxy-4-(3′,4′-dichlorophenyl or 4′-bromophenyl) hexanamides.
